(19) United States
(12) Patent Application Publication
(10) Pub. No.: US 2022/0125386 A1
(43) Pub. Date: Apr. 28, 2022

(54) SYSTEMS AND METHODS FOR IMPROVING CHRONIC CONDITION OUTCOMES USING PERSONALIZED AND HISTORICAL DATA

(71) Applicant: Ohio State Innovation Foundation, Columbus, OH (US)

(72) Inventors: William S. Marras, Powell, OH (US); Jonathan Dufour, Columbus, OH (US); Alexander Aurand, Columbus, OH (US); Prasath Mageswaran, Columbus, OH (US); Gregory Knapik, Columbus, OH (US)

(21) Appl. No.: 17/458,756

(22) Filed: Aug. 27, 2021

Related U.S. Application Data
(60) Provisional application No. 63/106,531, filed on Oct. 28, 2020.

(51) Int. Cl. A61B 5/00; A61B 5/11; G16H 10/60; G16H 50/20; G16H 10/20; G06N 20/00

(52) U.S. Cl. CPC A61B 5/7275; A61B 5/1116; G16H 10/60; A61B 2503/24; G16H 10/20; G06N 20/00; A61B 2562/0219; G16H 50/20

(57) ABSTRACT

An integrated and holistic system that delivers clinical decision support, disorder prevention, and research services for chronic disorders is provided. In one embodiment, the system collects a variety of data about an individual including data from one or more of wearable motion sensors, self-reported questionnaires, medical imaging, and electronic medical records. A historical database of outcomes and similar data for other individuals is processed using advanced statistics, artificial intelligence, and machine learning to identify biomarkers and phenotypes that are indicative of outcomes with respect to zero or more interventions. The collected individual's data is then analyzed with respect to the identified biomarkers or phenotypes to predict outcomes with respect to zero or more interventions for the individual. The individual, and/or an associated agent, may then consider the predicted outcomes when selecting an intervention plan for the individual and monitor intervention impact over time.

### CROSS-REFERENCE TO RELATED APPLICATIONS

This application claims priority to U.S. Provisional Patent Application No. 63/106,531 filed on Oct. 28, 2020, the disclosure of which is incorporated by reference in its entirety.

### STATEMENT OF GOVERNMENT SUPPORT

This invention was made in part with government support under W81XWH2010878, W81XWH20C0045, and W81XWH20C0007 awarded by Army Medical Research and Materiel Command, UH2AR076729 awarded by The National Institutes of Health, and N3239820P0600 awarded by Naval Medical Research Center. The government has certain rights in the invention.

### BACKGROUND

When patients seek medical care from medical providers such as doctors, the data that the medical providers use to inform their diagnosis or treatment recommendation is often limited to incomplete, subjective, and hard to access data. Employers face similar challenges when trying to prevent injuries from occurring in their workforce, as available information is generally limited to subjective and incomplete evaluations. Researchers who study these problems are similarly forced to rely on subjective data to make their conclusions. This lack of objective, quantitative, and actionable data, especially with respect to the diverse biopsychosocial factors associated with chronic disorders, may result in an incomplete picture of an individual's condition, risk for injury, or likelihood to respond positively to interventions.

### SUMMARY

An integrated and holistic system for providing clinical decision support to medical providers who treat chronic conditions, for helping employers assess occupational injury risk and prevent chronic disorders, and for empowering researchers to discover novel treatments, interventions, and risk factors for chronic disorders is provided. In one embodiment, the system collects a variety of data about an individual including data from one or more of wearable motion sensors, self-reported questionnaires, medical imaging, and electronic medical records. A historical database of outcomes and similar data for other individuals is processed using advanced statistics, artificial intelligence, and machine learning to identify biomarkers (a specific observable trait, characteristic, state, status, or feature of an individual) and phenotypes (a set of observable traits, characteristics, states, status, or features that make an individual unique) that are indicative of outcomes with respect to zero or more interventions. The collected individual's data is then analyzed with respect to the identified biomarkers or phenotypes to predict outcomes with respect to zero or more interventions for the individual. The individual, and/or an associated agent, may then consider the predicted outcomes when selecting an intervention plan for the individual. Additional features of the system may include the ability of agents to monitor the data collected about an individual or group of individuals over time to determine if the individual or group of individuals is complying with an intervention plan, to determine if outcomes are getting better or worse, and to determine the success of interventions.

In an embodiment, a method is provided. The method includes: receiving data associated with an individual by a computing device; applying a model to the individual's data to identify a phenotype associated with the individual by the computing device; making a prediction for the individual based on the identified phenotype by the computing device; and providing the prediction to the individual or an agent of the individual by the computing device.

Embodiments may include some or all of the following features. Receiving data may include receiving the individual's data from one or more sensors worn by the individual. The sensor may include one or more inertial measurement unit (IMU) sensors. Receiving data may include receiving medical history data for the individual. Receiving data may include receiving biopsychosocial biomarkers for the individual. Receiving data may include receiving data from one or more digital questionnaires completed by the individual. The identified phenotype may be derived from one or more biomarkers that is an indicator of dynamic low back motion function. The identified phenotype may be derived from one or more biomarkers that is an indicator of dynamic neck motion function. The prediction may include a predicted success likelihood for a medical procedure. The prediction may include an injury likelihood or injury for the individual. The prediction may include an injury likelihood for a group of individuals. The individual may be a patient or an employee. The method may further include: receiving a historical reference database comprising a plurality of records; for each record, identifying unique biomarkers associated with the record; and training the model using the plurality of records and biomarkers to identify unique phenotypes.

In an embodiment, a technology platform for providing patient care, injury prevention, or research services is provided. The platform includes at least one computing device and a computer-readable medium with computer-executable instructions stored thereon that when executed by the at least one computing device cause the at least one computing device to: receive data associated with an individual; apply a model to the individual's data to identify a phenotype associated with the individual; make a prediction for the individual based on the identified phenotype; and provide the prediction to the individual or an agent of the individual.

Embodiments may include some or all of the following features. The computer-executable instructions may include computer-executable instructions that when executed by the at least one computing device cause the at least one computing device to: receive the user data from one or more sensors worn by the individual. The sensor may include an inertial measurement unit (IMU) sensor. The received data may include medical history data for the individual. The received data may include data from one or more digital questionnaires completed by the individual. The prediction may include a predicted success likelihood for a medical procedure. The prediction may include an injury likelihood or injury risk for the user.

### BRIEF DESCRIPTION OF THE DRAWINGS

The following detailed description of illustrative embodiments is better understood when read in conjunction with the appended drawings. For the purpose of illustrating the embodiments, there is shown in the drawings example constructions of the embodiments; however, the embodiments are not limited to the specific methods and instrumentalities disclosed. In the drawings:

FIG. 1 is an illustration of how the technology platform or system allows users to capture data and provide insights to inform patient care, injury prevention, and research;

FIG. 2 is an illustration demonstrating that the technology platform is a system or family of modules or applications that the user can access to address different needs or use cases;

FIG. 3 is an illustration of the various types of inputs and outcomes the technology platform leverages, as well as the various types of potential customers or users;

FIG. 4 is an illustration of an example of the technology platform architecture;

FIG. 5 is an illustration of an example general data flow for some example types of data;

FIG. 6 is an illustration of example wearable sensors, harnesses, and transportation and charging station;

FIG. 7 is an illustration of an example informed patient treatment workflow;

FIG. 8 is an illustration of an example informed injury prevention workflow;

FIG. 9 is an illustration of an example informed research workflow;

FIG. 10 is an illustration of an example processing flowchart for an example mechanistic biomechanical model;

FIG. 11 is an illustration of some example three-dimensional visualizations for some example mechanistic biomechanical models.

FIG. 12 is an illustration of an example low back motion assessment protocol that is used to assess spine motion capabilities such as flexibility, speed, acceleration, symmetry, fluidity, and consistency via Inertial Measurement Unit (IMU) sensors;

FIG. 13 is an illustration of an example functional neck motion assessment protocol that is used to assess spine motion capabilities such as flexibility, speed, acceleration, symmetry, fluidity, and consistency via inertial measurement unit (IMU) sensors;

FIG. 14 is an illustration of an example functional low back motion assessment data collection workflow;

FIG. 15 is an illustration of an example of a digital questionnaire data collection workflow;

FIG. 16 is an illustration of an example project summary report dashboard that helps users track subject enrollment and demographics;

FIG. 17 is an illustration of an example individual summary report dashboard that helps users track event completion progress and overall subject journey;

FIG. 18 is an illustration of an example individual summary report dashboard showing processed feature and characteristic measurements for motion capabilities and pain;

FIG. 19 is an illustration of an example individual summary report dashboard showing composite biomarkers, as well as normalized (e.g., t-scores) feature and characteristics for motion capabilities, pain, and other biopsychosocial biomarkers;

FIG. 20 is an illustration of an example individual summary report dashboard showing composite biomarker percentiles and modeled treatment outcome probabilities;

FIG. 21 is an illustration of an example population summary report dashboard showing differences between cohorts for specific motion assessment feature biomarkers; and

FIG. 22 is an operational flow of an implementation of a method for generating one or more predictions based on user data.

### DETAILED DESCRIPTION

The following presents a simplified overview of the example embodiments in order to provide a basic understanding of some aspects of the example embodiments. This overview is not an extensive overview of the example embodiments. It is intended to neither identify key or critical elements of the example embodiments nor delineate the scope of the appended claims. Its sole purpose is to present some concepts of the example embodiments.

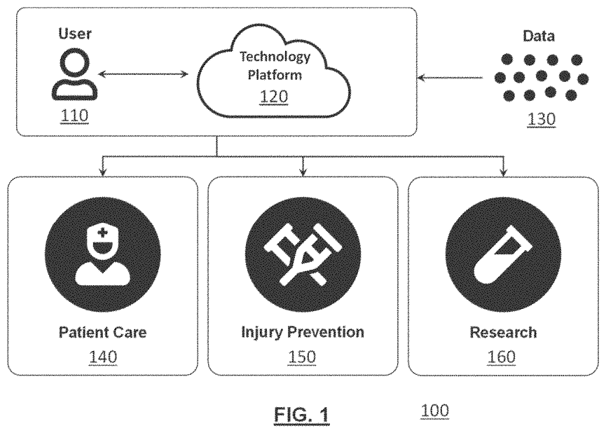

FIG. 1 is an illustration of an exemplary environment 100 where users 110 leverage the system, herein called technology platform 120, to capture data 130 from one or more individuals for the purpose of informed patient care 140, injury prevention 150, and/or research 160. The received data 130 may include data relevant to a particular chronic medical condition associated with an individual such as back pain, neck pain, shoulder pain, other joint pain, musculoskeletal disorder, diabetes, heart disease, anxiety, depression, other psychological disorder, or cancer. Other medical conditions may also be supported.

Data 130 may include cross-sectional, prospective, and retrospective records. Data may be captured within the context of standard business activities, healthcare activities, personal activities, or as part of experimental or observational studies.

Patient care applications 140 include workflows that allow users 110 to objectively assess patient condition, access a holistic suite of biopsychosocial biomarkers, track patient progress over time, identify unique patient phenotypes to support diagnoses, predict treatment outcomes, evaluate treatment effectiveness, support informed treatment decision making, identify best practices for specific patient populations or phenotypes, determine value-based reimbursement, and facilitate provider to patient communication.

Injury prevention applications 150 include occupational and personal workflows that allow users 110 to identify risk factors that drive injury risk, prioritize prevention resources, inform design of engineering controls to mitigate risk, evaluate intervention impact, return injured employees to work safely, promote a culture of occupational safety, and facilitate employer to employee communication.

Research applications 160 include workflows that allow users 110 to implement research from start to finish, design studies, collect data, perform analyses, visualize results, create reports, manage research operations, meet regulatory and cybersecurity requirements, and scale research through automation.

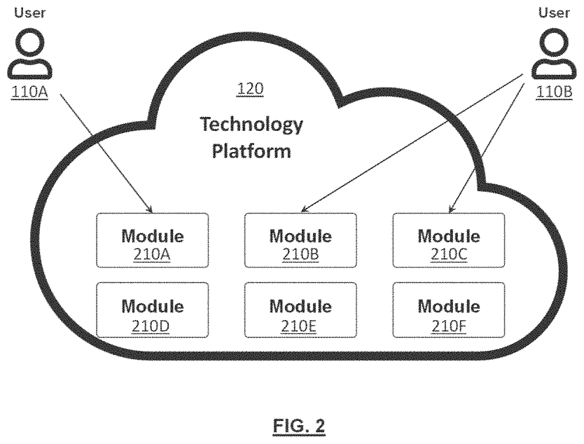

FIG. 2 illustrates that the technology platform 120 is comprised of a series of modules 210 (e.g., the modules 210A-210F) that the user 110 (e.g., the users 110A and 110B) interacts with. Modules 210 serve as applications and provide the user 110 access to specific functionality and interfaces within the technology platform 120. Modules 210 may interact with each other or operate independently within the technology platform 120.

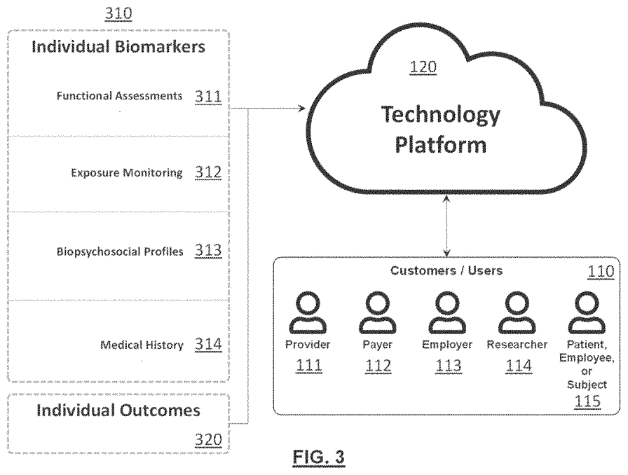

FIG. 3 illustrates the type of data that may be collected into the technology platform 120 and the types of users 110 (e.g., provider 111, payer 112, employer 113, researcher 114, and individual patient, employee, or subject 115 who may use it.

Collected data may include individual biomarkers 310 that are generally used as inputs to predict or provide context to a specific condition, as well as individual outcomes 320 that are generally the endpoint of interest that users 110 are trying to diagnose, treat, prevent, or generally improve. Note that some data may serve as both individual biomarkers 310 and individual outcomes 320.

Individual biomarkers 310 may be derived from functional assessments 311, exposure monitoring 312, biopsychosocial profiles 313, and/or medical history 314. Other categories of individual biomarkers 310 may also be included.

Functional assessments 311 are standardized evaluations of an individual's function, typically via wearable sensors 410. These assessments generally require individuals to perform a standardized protocol while data is recorded by one or more wearable sensors 410. Wearable sensors 410 may include sensors designed to capture motion or kinematics, general activity, muscle activity, heart activity, brain activity, sleep, oxygenation, mechanical force, or temperature. Note that the term wearable sensors 410 is used generally throughout this document, however, some included solutions may not necessarily be wearable (e.g., a markerless motion capture camera system). Other types of sensors may also be included. The wearable sensors 410 may be self-contained or may be part of another device such as a smartphone or smartwatch worn or carried by the patient, employee, or subject 115.

Exposure monitoring 312 applications are evaluations of occupational and life exposures via wearable sensors 410 and other digital measurement techniques (e.g., digital questionnaires). These assessments generally require individuals to perform their regular occupational duties or activities of daily living while data is recorded by the wearable sensors 410 and other digital measurement techniques to evaluate exposures experienced during these activities. Wearable sensors 410 may include sensors designed to capture motion, kinematics, general activity, muscle activity, heart activity, brain activity, sleep, oxygenation, mechanical force, or temperature, and may not necessarily be worn as described previously herein. Other types of sensors may also be included.

Biopsychosocial profiles 313 use digital questionnaires that are filled out by individual patients, employees, or subjects 115 to capture a holistic array of biopsychosocial contributors. Questionnaires can be taken in person on a computing device or via email, text, or other digital delivery mechanisms. Questionnaires may be custom or derived from existing validated sources. Questionnaires may be completed once, periodically (e.g., daily, or weekly), or on a specific schedule (e.g., baseline, 3-month follow-up, 1-year follow-up). In the event that the patient, employee, or subject 115 does not complete the questionnaire, the cloud web application may send periodic reminders to the patient, employee, or subject 115. Questionnaires may be configured or selected by the user 110 and may investigate domains of general health, pain, stiffness, injury, activity, exercise, physical function, disability, anxiety, depression, fear avoidance, self-efficacy, sleep, fatigue, social support, family support, resilience, personality, preferences, beliefs, employment, lost work time, substance use, medication use, opioid use, medical history, treatment history, and demographics. Other domains may also be included, and similar biopsychosocial profile 313 data may be captured through other sources (e.g., medical records, omics, imaging, etc.).

Medical history 314 assessments evaluate past or current relevant medical history through manual, software-assisted, or fully automated transcription of electronic health records. Depending on the embodiment, the patient, employee, or subject 115 may authorize the technology platform 120 to request data related to their medical history 314 from one or more medical providers. Information captured may include diagnoses, treatments, biomedical imaging, and other clinical tests. Biomedical imaging data may include one or more medical images of studies taken of the patient, employee, or subject 115 such as X-rays, CT scans, MRI scans, or ultrasounds. Other types of information and imaging may also be included.

Individual outcomes 320 that are targeted as endpoints may include pain, stiffness, injury, claims, healthcare utilization, activity, exercise, physical function, disability, anxiety, depression, fear avoidance, self-efficacy, sleep, fatigue, resilience, employment, lost work time, productivity, substance use, medication use, and opioid use. Other outcomes may also be included.

Users 110 of the technology platform 120 may include a diverse array of healthcare providers 111, healthcare payers 112, employers 113, researchers 114, and patients, employees, or subjects 115. Other users 110 may also leverage the technology platform 120.

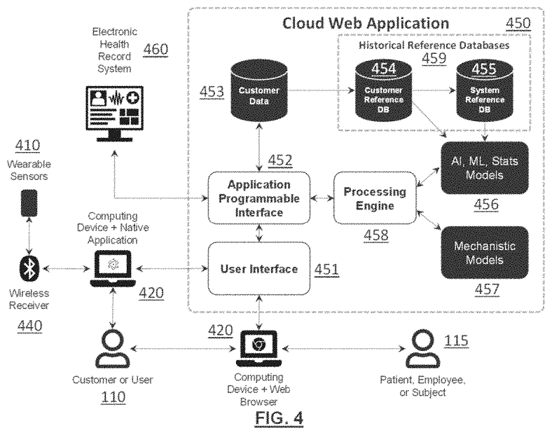

FIG. 4 illustrates an example of the technology platform 120 architecture. In this architecture, the user 110 captures data from wearable sensors 410 or from an individual patient, employee, or subject 115 through a computing device 420 that is connected to one or more user interfaces 451 hosted in a cloud web application 450. A wireless receiver 440 may be used to bridge communication between the wearable sensors 410 and the computing device 420.

The one or more user interfaces 451 leverage an application programmable interface 452 to read, write, and modify customer data. The application programmable interface 452 can also be used to access data from and supply results to electronic health record systems 460 or other approved third-party applications.

Customer data is stored in one or more cloud customer databases 453. Some data from the one or more customer databases 453 may be added to the one or more historical customer reference databases 454. Data from one or more historical customer reference databases 454 may be accumulated in one or more historical system reference databases 455. Together, these historical customer reference databases 454 and system reference databases 455 are referred to herein as historical reference databases 459. Historical reference databases 459 may be anonymized such that the individual associated with any particular record cannot be identified.

Artificial intelligence, machine learning, and advanced statistical models 456 leverage the historical reference databases 459 to support analysis of customer data through a processing engine 458 that operates as the central processing and interpretation unit for the technology platform 120.

Artificial intelligence, machine learning, and advanced statistical models 456 may leverage traditional statistical methods, as well as both supervised and unsupervised learning methods to classify individual patients, employees, or subjects 115 into specific subgroups or phenotypes 710 (e.g., 710A, 710B, 710C). These phenotypes 710 may be used to further classify specific conditions or to associate a specific set of traits and characteristics with specific outcomes. Models may be created from a static snapshot of reference data 459 at a given point in time, or may change continuously as new data is fed into the technology platform 120.

The processing engine 458 may also utilize mechanistic models 457 to analyze customer data, which will be discussed in detail later.

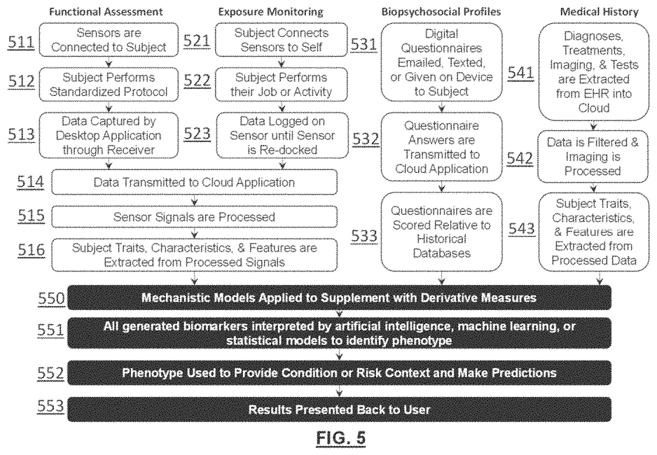

FIG. 5 illustrates an example of how data 130 transitions from its raw collection source to results that are presented back to the user.

For functional assessments 311, wearable sensors 410 are connected at 511 to the individual patient, employee, or subject 115. The individual patient, employee, or subject, 115 then performs a standardized protocol at 512. Data is captured at 513 by a computing device 420 through a wireless receiver 440. Data is then transferred at 514 to the cloud web application 450 where wearable sensor 410 signals are processed at 515 and individual patient, employee, or subject 115 traits, characteristics, and features are extracted at 516 by the processing engine 458.

For exposure monitoring 312, the individual patient, employee, or subject 115 connects the wearable sensors 410 to herself at 521. The individual patient, employee, or subject 115 then performs her job or activity of interest at 522 while data is logged on the wearable sensors 410 at 523. Once the wearable sensors 410 are docked, data is then transferred at 514 to the cloud web application 450 where wearable sensor 410 signals are processed at 515 and individual patient, employee, or subject 115 traits, characteristics, and features are extracted at 516 by the processing engine 458.

For biopsychosocial profile questionnaires 313, questionnaires are emailed texted or given on a device at 531 to the individual patient, employee, or subject 115. As questions are answered, answers are transmitted at 532 to the cloud web application 450 where they are scored at 533 relative to historical reference databases 459 by the processing engine 458.

For medical history 314, diagnoses, treatments, imaging, tests, and other data are extracted from an individual patient, employee, or subject's 115 electronic health record 460 and transmitted at 541 to the cloud web application 450. Received data is filtered and imaging is processed at 542 by the processing engine 458. The processing engine 458 then extracts traits, characteristics, and features from the received data at 543.

For all of functional assessments 311, exposure monitoring 312, biopsychosocial profiles 313, and medical history 314 data, data may be further processed via mechanistic biomechanical models at 550 to generate additional biomarkers 310. All generated biomarkers 310 may be interpreted by artificial intelligence, machine learning, and/or advanced statistical models 456 to identify an individual patient, employee, or subject's, 115 unique phenotype 710 at 551. This unique phenotype 710 may then be used to provide condition or risk context and make outcome predictions 552. Results are then presented 570 back to the user 110 at 553.

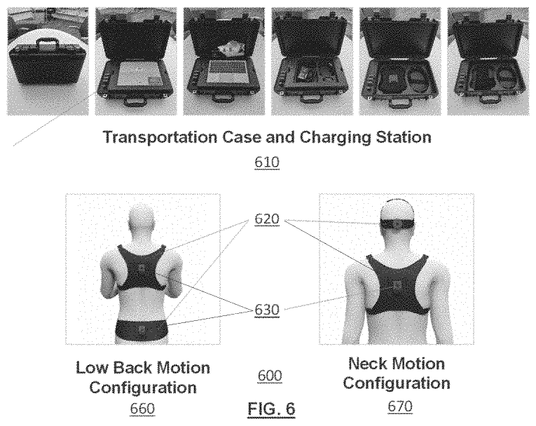

FIG. 6 illustrates an example of a wearable sensor 410 hardware system 600 that may be used by the user 110 and the technology platform 120. Wearable sensor hardware systems 600 may include a transportation case and charging station 610, which may also operate as a docking station to transmit data from sensors to the cloud web application 450. Hardware systems may also include wearable harnesses 620, sensors 630, and receivers 640. Data is typically stored temporarily on the sensor 630 itself or in temporary memory on a computing device 420 before being sent to the cloud web application 450.

In embodiments directed to back, neck, or other musculoskeletal medical conditions, sensor data may be received from one or more inertial measurement unit (IMU) sensors placed on the body of the patient, employee, or subject 115. Other sensor types and placements may also be used.

In one example, with respect to lower-back pain and low back motion assessments 660, the patient, employee, or subject 115 may wear a vest that includes a first inertial measurement unit sensor 630 on their back or any other location that enables tracking of the ribcage or other body segment immediately above the top of the lumbar spine and a second inertial measurement unit sensor 630 on a belt on their waist or any other location that enables tracking of the pelvis or other body segment immediately below the lumbar spine. The first and second inertial measurement unit sensors 630 may generate sensor data including their rotational and linear positions, velocities, and accelerations that may be received by the technology platform 120. Sensors 630 may also be placed directly on the patient, employee, or subject's 115 skin or clothing. Other similar sensing systems that capture motion may also be used in addition to or in place of the inertial measurement unit sensors (e.g., a markerless motion capture system). Some implementations may also only use one inertial measurement unit sensor placed on the back or any other location that enables tracking of the ribcage or other body segment immediately above the top of the lumbar spine.

In another example, with respect to neck pain and neck motion assessments 670, the patient, employee, or subject 115 may wear a vest that includes a first inertial measurement unit sensor 630 on their back or any other location that enables tracking of the ribcage or other body segment immediately below the bottom of the cervical spine and a second inertial measurement unit sensor 630 (not shown) on the front of a headband on their head or any other location that enables tracking of the skull or other body segment immediately above the cervical spine. The first and second inertial measurement unit sensors 630 may generate sensor data including their rotational and linear positions, velocities, and accelerations that may be received by the technology platform 120. Other similar sensing systems that capture motion may also be used in addition to or in place of the inertial measurement unit sensors (e.g., a markerless motion capture system). Some implementations may also only use one inertial measurement unit sensor placed on the head or any other location that enables tracking of the skull or other body segment immediately above the top of the cervical spine.

In some embodiments, with respect to wearable sensors 410, the patient, employee, or subject 115 may continuously wear or carry the wearable sensors 410. For example, the patient, employee, or subject 115 may be instructed to always wear an activity monitor watch. In other embodiments, the patient, employee, or subject 115 may be instructed to wear the wearable sensors 410 for some predetermined amount of time or during certain activities. For example, the patient, employee, or subject 115 may be asked to wear or carry the wearable sensors 410 while performing their typical occupational duties such as during manual materials handling or specific activities of daily living such as exercising or sleeping.

Example wearable sensor and harness configurations for Low Back Motion 660 and Neck Motion 670 evaluations are for example only. Other wearable sensor and harness configurations may also be used.

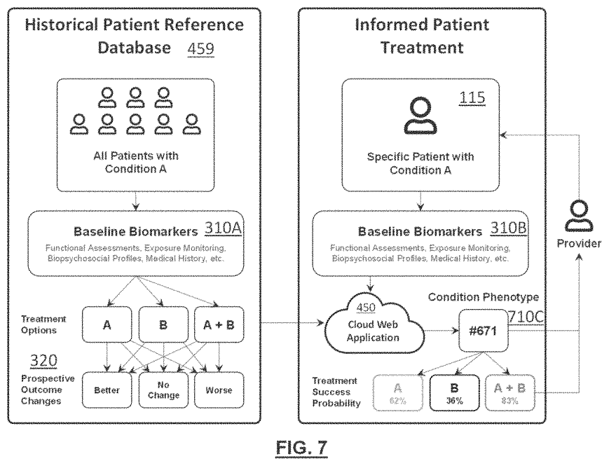

FIG. 7 illustrates an example informed patient care use case for the technology platform 120. In this example, a historical reference database 459 is created that includes baseline assessments of individual biomarkers 310 (e.g., biomarkers 310A and 310B) for a large quantity of individual patients, employees, or subjects 115 suffering from a specific medical condition or family of medical conditions (e.g., low back pain). Following the baseline assessments, individual patients, employees, or subjects 115 are tracked prospectively to observe which treatments they receive and whether their individual outcomes 320 get better, do not change, or get worse. This historical reference database 459 is then used by the cloud web application 450 to identify a series of unique phenotypes (a unique grouping of traits or characteristics) 710C that are associated with positive or negative prospective outcome changes 320 relative to one or more specific treatment options.

After unique phenotypes 710C have been identified, the cloud web application 450 determines which unique phenotype or phenotypes 710C a specific new patient, employee, or subject 115 belongs to based on a baseline assessment of the patient, employee, or subject's 115 individual biomarkers 310. By identifying which phenotype 710C a patient, employee, or subject 115 belongs to, treatment success probabilities can be estimated based on observations of treatment response for individual patients, employees, or subjects 115 with that same phenotype or phenotypes 710C in the historical reference database 459. These estimates can then be used by the provider 111 and the patient, employee, or subject 115 to determine the best course of treatment for that individual patient, employee, or subject 115.

In addition to identifying unique phenotypes 710C and predicting treatment success outcome probabilities, the technology platform 120 can also be used to identify and operationalize novel objective composite biomarkers that are indicative of a specific condition state, nature, severity, or outcome. The advantage of composite biomarkers is that they can incorporate inputs from multiple biopsychosocial domains. Additionally, the ability of the technology platform 120 to reference novel biomarkers to large reference databases helps provide intuitive real-time context to support users in their interpretation of meaningful thresholds and meaningful changes over time.

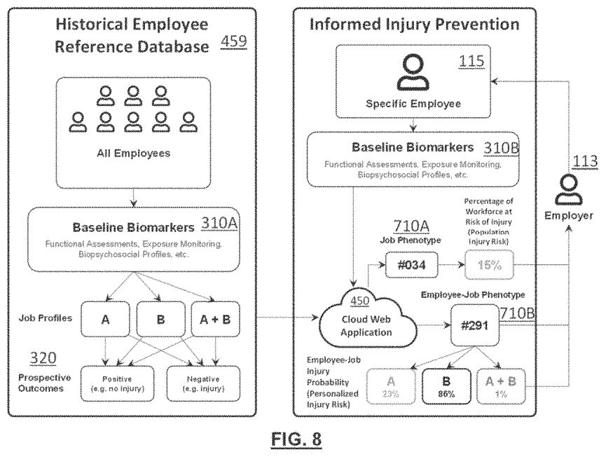

FIG. 8 illustrates an example informed injury prevention use case for the technology platform 120. In this example, a historical employee reference database 459 is created that includes baseline assessments of individual biomarkers 310 (e.g., biomarkers 310A and 310B) for a large quantity of employees at risk for a specific medical condition or family of medical conditions (e.g., neck pain). Following the baseline assessments, employees are tracked prospectively to observe which jobs they perform and whether their individual outcomes 320 are positive (e.g., no injury) or negative (e.g., injury). This historical employee reference database 459 is then used by the cloud web application 450 to identify a series of unique job and employee-job phenotypes 710 (e.g., phenotypes 710A and 710B, respectively) that are associated with positive or negative prospective outcomes 320.

After unique phenotypes 710 have been identified, the cloud web application 450 determines which unique employee-job phenotype or phenotypes 710 a specific new employee 115 belongs to based on a baseline assessment of the employee's 115 individual biomarkers 310. By identifying which phenotype 710 an employee 115 belongs to, an individual's injury probabilities (i.e., personalized injury risk) can be estimated based on observations of injuries for employees with that same employee-job phenotype or phenotypes 710 in the historical employee reference database 459. If only job-specific information is available, estimates of the percentage of workforce at risk for injury (population injury risk) can still be estimated. These estimates can then be used by the employer 113 and the employee 115 to determine the best course of workplace intervention for that individual employee 115.

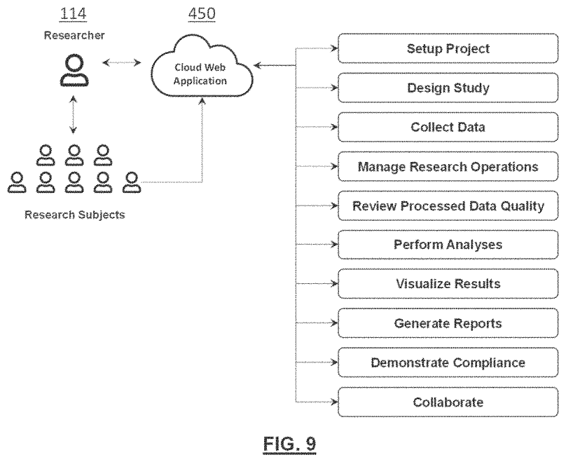

FIG. 9 illustrates an example research use case for the technology platform 120. In this example, a researcher 114 leverages the cloud web application 450 to execute research projects from start to finish. The technology platform 120 may include features that allow a researcher 114 to setup projects, design studies, collect data, manage research operations, review processed data quality, perform analyses, visualize results, generate reports, demonstrate regulatory and cybersecurity compliance, and collaborate with colleagues across the world. Other features and capabilities may also be supported.

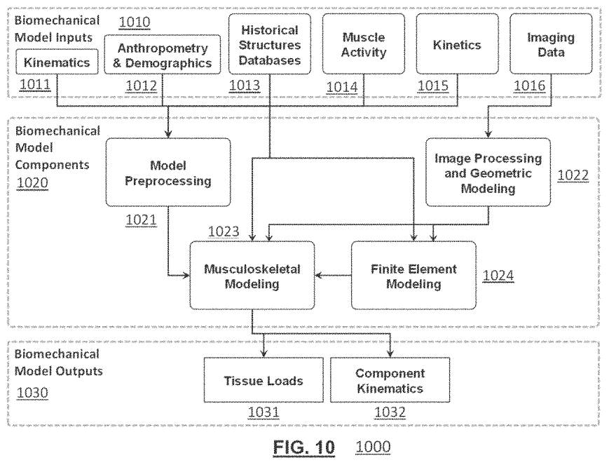

FIG. 10 illustrates an example processing flowchart for an example mechanistic biomechanical model 1000. The primary purpose of a mechanistic biomechanical model 1000 is to estimate dynamic signals, features, or characteristics (biomechanical model outputs 1030) of an individual patient, employee, or subject 115 that cannot be directly measured (e.g., forces on internal spine tissues) while performing standardized protocols, occupational activities, or activities of daily living. To quantify these unmeasurable signals, features, or characteristics, mechanistic biomechanical models 1000 capture signals, features, or characteristics (biomechanical model inputs 1010). These measurable biomechanical model inputs 1010 are then processed by a series of customizable biomechanical model components 1020 that are designed to consider how the biomechanical model inputs 1010 interact with each other and influence the overall system to produce the biomechanical model outputs 1030 of interest. A three-dimensional computer model is typically, but not always, generated to support computations and help visualize results. More or fewer biomechanical model components 1020 may be supported, and each biomechanical model component 1020 may consist of a series of sub biomechanical model components 1020. Some or all of the biomechanical model components 1020 may be implemented together or separately.

Biomechanical model inputs 1010 can be obtained from a variety of sources and typically help quantify specific characteristics or features that make an individual patient, employee, or subject 115 unique. Other types of biomechanical model inputs 1010 may also be supported in addition to those defined below.

Biomechanical model inputs derived from kinematics 1011 may include data from an individual patient, employee, or subject 115 from optical motion capture systems, inertial measurement systems, magnetic tracking systems, ultrasonic measurement systems, and/or goniometric systems. Other motion sensor systems may also be used. These systems may capture the motion or kinematics of an individual's entire body or may focus specifically on a subset of body joints (e.g., cervical spine, lumbar spine, shoulder, or knee) or segments (e.g., pelvis, head, ribcage, or thigh). The motion of tools, job implements, workstation elements, assistive devices, and/or medical devices may be captured at the same time with these systems. The motion of other objects may be captured as well.

Biomechanical model inputs derived from anthropometry and demographics 1012 may include the individual patient, employee, or subject's 115 height, weight, age, and/or measurements of individual body segments (e.g., chest circumference, arm length, etc.). Other body measures may be included as well. Anthropometric measures may be recorded with anthropometers, calipers, stadiometers, tape measures, scales, optical scanners, laser scanners, or any of the kinematic measurement systems mentioned above. Other measurement devices may be used as well.

Biomechanical model inputs derived from historical structures databases 1013 may include data describing bone or soft tissue geometry, composition, or material properties. Databases may be commercially available or custom to this technology platform 120. Sources of data may include biomedical images, structure geometry, structure models, and/or material properties data. Other types of data sources may be included.

Biomechanical model inputs derived from muscle activity 1014 include raw and processed muscle activity data for one or more muscles captured from electromyography (EMG), acoustic myography, or other muscle activity sensing technology. Muscle activities may be captured from an individual patient, employee, or subject 115, or may be derived from historical reference databases 459 that are commercially available or custom to this technology platform 120.

Biomechanical model inputs derived from kinetics 1015 may include data captured from pressure sensors, load cells, force plates, and/or other sensors that measure forces, torques, moments, and/or pressure. Other types of sensors may be included. Measured forces may include forces applied to an individual patient, employee, or subject 115 or forces applied to other relevant objects (e.g., tools, equipment, or the ground).

Biomechanical model inputs derived from imaging data 1016 may include one or more medical images of studies taken from a patient, employee, or subject 115 such as X-rays, CT scans, MRI scans, or ultrasounds. Other types of imaging technologies may also be included.

Biomechanical model components 1020 generally consist of one or more software functions, algorithms, applications, and/or programs that are designed to process data and aid in transforming biomechanical model inputs 1010 into biomechanical model outputs 1030. While the majority of the biomechanical model components 1020 are custom and developed specifically to support the defined one or more biomechanical models 1000, they may also leverage commercial software applications, libraries, packages, functions, or programs. While biomechanical model components 1020 are typically software by nature, in some cases they may also include hardware (e.g., electrical components that transform signals) or firmware (e.g., software embedded on a micro-computer).

Biomechanical model outputs 1030 are the signals, characteristics, features, or other data transformed by the biomechanical model components 1020 and made available to the user 110. Biomechanical model outputs 1030 typically are more descriptive, predictive, or conceptually meaningful than the biomechanical model inputs 1010 on their own. Other types of biomechanical model outputs 1030 may also be supported in addition to those defined below.

Tissue loads 1031 are the calculated forces, moments, torques, stresses, pressures, and/or other measures of mechanical load on various model elements including bones (e.g., vertebral bodies), intervertebral discs, muscles, ligaments, tendons, and nerves. Mechanical loads may also be calculated on internal non-body objects such as surgical screws, rods, plates, cages, inserts, and/or external objects such as tools, job implements, workstation elements, assistive devices, and/or medical devices. Mechanical loads may be calculated for other modeled elements as well.

Component kinematics 1032 are the calculated kinematic outputs measured from various mechanistic biomechanical model 1000 elements. Component kinematic 1032 outputs may include refined calculated motions of external body elements such as the arms, legs, head, and trunk, as well as internal body elements such as bones (e.g., vertebral bodies), intervertebral discs, muscles, ligaments, tendons, and nerves. Outputs may include calculated measures of element rotational and translational positions, velocities, and/or accelerations. They may also include other motion-related measures such as strains, centers of rotation, and clearance. Other body elements and measures may be included.

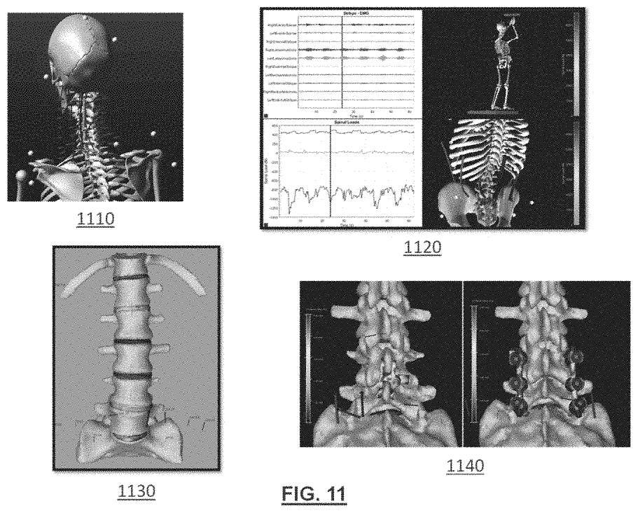

FIG. 11 illustrates some example mechanistic biomechanical models 1000. Other mechanistic biomechanical models 1000 may also be supported in addition to those defined below.

In one example image 1110, a mechanistic biomechanical model 1000 of the cervical spine that is used to evaluate neck disorder risk during occupational work is shown. This model is developed from an individual patient, employee, or subject's 115 anthropometry and demographic measures 1012 including height, weight, and age, CT imaging data 1016, and neck musculature data from a historical structures database 1013. Optical motion capture data is used as kinematic inputs 1011 and surface electromyography data is used for muscle activity 1014 inputs. The example image 1110 shows the modeled skeleton structure, musculature, and optical motion capture markers derived from the biomechanical model components 1020 and biomechanical model inputs 1010 captured while the patient, employee, or subject 115 performs one or more specific occupational tasks. From this model, intervertebral disc and neck musculature forces (i.e., tissue loads 1031) are produced as outputs to in order to identify the source and likelihood (by comparing to known tissue injury thresholds) of injury risk so that the workstation can be modified to make it safer and prevent future injuries.

In another example 1120, a mechanistic biomechanical model 1000 of the lumbar spine that is used to develop safe guidelines for overhead occupational tasks is shown. This model is developed and executed in a laboratory research setting, but the results can be translated into simple and effective guidelines that can be applied in practice within occupational work environments. This model is developed from various subject anthropometry and demographic 1012 measures including height, weight, age, and several torso measurements. Musculature and bone data from historical structures databases 1013 are used to construct the model low back musculature and rest of the skeleton. Optical motion capture data is used as kinematic inputs 1011, force plate and load cell data is used as kinetic inputs 1015 to quantify external loads on the body, and surface electromyography data is used to quantify muscle activities 1014. The example image 1120 shows an example plot of electromyography data, a graphical representation of the individual's entire body while performing a specific occupational task, a graphical representation of a zoomed in view of the individual's lumbar spine, and a plot of the calculated forces on the intervertebral discs of the spine during the entire task.

In another example 1130, a mechanistic biomechanical model 1000 of the lumbar spine that is used to better understand a patient, employee, or subject's 115 specific spine condition is shown. This model is developed from CT and MRI imaging data 1016 and historical structures databases 1013 of tissue material properties and ligament locations. Kinematic data 1011 captured from inertial measurement unit sensors and muscle activities 1014 captured from surface electromyography sensors also serve as inputs. Motion and electromyography data are filtered and pre-processed 1021. CT and MRI data is processed and then transformed 1022 into personalized spine geometry. Finite element modeling 1024 is used to represent the intervertebral discs. Components and inputs are then combined into a musculoskeletal model 1023 that calculates intervertebral disc stresses, ligament forces, and facet joint tissue loads 1031, as well as intervertebral component kinematics 1032. This data is then compared to historic data and may be used in additional machine learning models to quantify the individual patient, employee, or subject's 115 spine health, inform diagnoses, and inform treatment decisions. The example image 1130 shows the modeled lumbar spine including the vertebrae bones, the intervertebral discs with shading to represent stresses throughout the tissue, and force vector arrows to represent muscle, ligament, and bony contact forces.

In another example 1140, a mechanistic biomechanical model 1000 of the lumbar spine that is used to pre-operatively assess potential surgical outcomes is shown. This model is developed from CT and MRI imaging data 1016 and historical structures databases 1013 of tissue material properties, ligament locations, and surgical devices. Kinematic data 1011 captured from inertial measurement unit sensors and muscle activities 1014 captured from surface electromyography sensors also serve as inputs. CT and MRI data is processed and then transformed 1022 into personalized spine geometry. Finite element modeling 1024 is used to represent the intervertebral discs and surgical hardware. Components and inputs are then combined into a musculoskeletal model 1023 that calculates intervertebral disc stresses, ligament forces, and facet joint tissue loads 1031, as well as intervertebral component kinematics 1032 and stresses within each of the surgical screws, rods, and plates. The example image 1140 shows the modeled lumbar spine including the vertebrae bones, the intervertebral discs with shading to represent stresses throughout the tissue, stress distributions in the surgical constructs, and force vector arrows to represent muscle, ligament, and bony contact forces. Two different surgical constructs are examined, and results are compared to assess which procedure is most likely to be successful for the specific patient, employee, or subject 115. Similar models may be used to assess the impact of a surgical method on a population of individual patients, employees, or subjects 115, as well or to evaluate the efficacy of new medical devices.

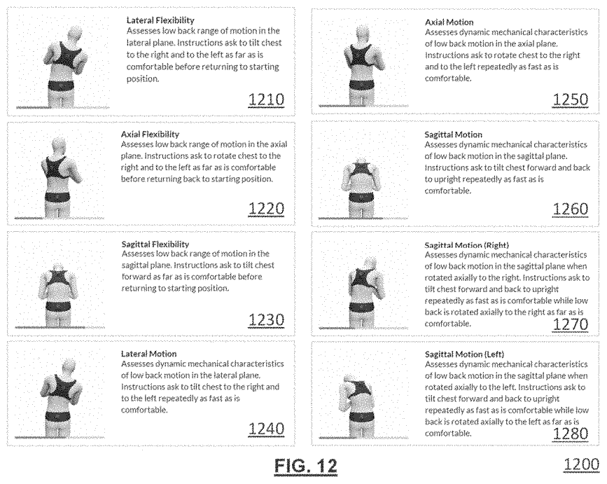

FIG. 12 illustrates a unique low back motion assessment protocol 1200 that is designed to assess an individual patient, employee, or subject's 115 three-dimensional low back or lumbar spine motion function or capabilities. This protocol is performed while the individual patient, employee, or subject 115 wears the low back motion assessment 660 sensor and harness configuration shown in FIG. 6 and described in previous sections. Specific motions that may be included in this protocol are described further below.

The low back lateral flexibility 1210 trial is used to assess low back range of motion in the lateral plane. For this lumbar spine motion, the individual patient, employee, or subject 115 is instructed to tilt their chest to the right and to the left as far as is comfortable before returning to the starting position.

The low back axial flexibility 1220 trial is used to assess low back range of motion in the axial plane. For this lumbar spine motion, the individual patient, employee, or subject 115 is instructed to rotate their chest to the right and to the left as far as is comfortable before returning to the starting position.

The low back sagittal flexibility 1230 trial is used to assess low back range of motion in the sagittal plane. For this lumbar spine motion, the individual patient, employee, or subject 115 is instructed to tilt their chest forward and back as far as is comfortable before returning to the starting position.

The low back lateral motion 1240 trial is used to assess dynamic mechanical characteristics of low back motion in the lateral plane. For this lumbar spine motion, the individual patient, employee, or subject 115 is instructed to tilt their chest to the right and to the left repeatedly as fast as is comfortable.

The low back axial motion 1250 trial is used to assess dynamic mechanical characteristics of low back motion in the axial plane. For this lumbar spine motion, the individual patient, employee, or subject 115 is instructed to rotate their chest to the right and to the left repeatedly as fast as is comfortable.

The low back sagittal motion 1260 trial is used to assess dynamic mechanical characteristics of low back motion in the sagittal plane. For this lumbar spine motion, the individual patient, employee, or subject 115 is instructed to tilt their chest forward and back repeatedly as fast as is comfortable.

The low back sagittal motion (right) 1270 trial is used to assess dynamic mechanical characteristics of low back motion in the sagittal plane when rotated axially to the right. For this lumbar spine motion, the individual patient, employee, or subject 115 is instructed to tilt their chest forward and back repeatedly as fast as is comfortable while their low back is rotated axially to the right as far as is comfortable.

The low back sagittal motion (left) 1280 trial is used to assess dynamic mechanical characteristics of low back motion in the sagittal plane when rotated axially to the left. For this lumbar spine motion, the individual patient, employee, or subject 115 is instructed to tilt their chest forward and back repeatedly as fast as is comfortable while their low back is rotated axially to the left as far as is comfortable.

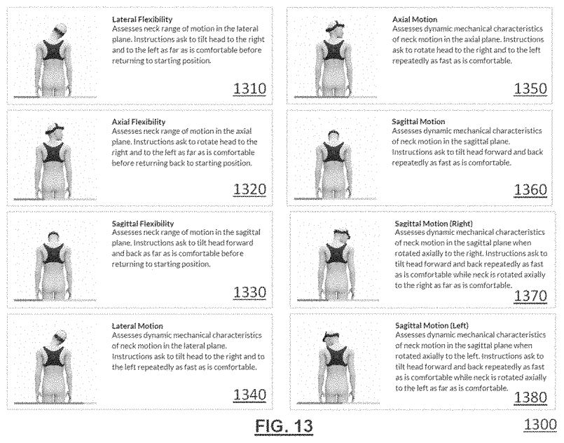

FIG. 13 illustrates a unique neck motion assessment protocol 1300 that is designed to assess an individual patient, employee, or subject's 115 three-dimensional neck or cervical spine motion function or capabilities. This protocol is performed while the individual patient, employee, or subject 115 wears the neck motion assessment 670 sensor and harness configuration shown in FIG. 6 and described in previous sections. Specific motions that may be included in this protocol are described further below.

The neck lateral flexibility 1310 trial is used to assess neck range of motion in the lateral plane. For this cervical spine motion, the individual patient, employee, or subject 115 is instructed to tilt their head to the right and to the left as far as is comfortable before returning to the starting position.

The neck axial flexibility 1320 trial is used to assess neck range of motion in the axial plane. For this cervical spine motion, the individual patient, employee, or subject 115 is instructed to rotate their head to the right and to the left as far as is comfortable before returning to the starting position.

The neck sagittal flexibility 1330 trial is used to assess neck range of motion in the sagittal plane. For this cervical spine motion, the individual patient, employee, or subject 115 is instructed to tilt their head forward and back as far as is comfortable before returning to the starting position.

The neck lateral motion 1340 trial is used to assess dynamic mechanical characteristics of neck motion in the lateral plane. For this cervical spine motion, the individual patient, employee, or subject 115 is instructed to tilt their head to the right and to the left repeatedly as fast as is comfortable.

The neck axial motion 1350 trial is used to assess dynamic mechanical characteristics of neck motion in the axial plane. For this cervical spine motion, the individual patient, employee, or subject 115 is instructed to rotate their head to the right and to the left repeatedly as fast as is comfortable.

The neck sagittal motion 1360 trial is used to assess dynamic mechanical characteristics of neck motion in the sagittal plane. For this cervical spine motion, the individual patient, employee, or subject 115 is instructed to tilt their head forward and back repeatedly as fast as is comfortable.

The neck sagittal motion (right) 1370 trial is used to assess dynamic mechanical characteristics of neck motion in the sagittal plane when rotated axially to the right. For this cervical spine motion, the individual patient, employee, or subject 115 is instructed to tilt their head forward and back repeatedly as fast as is comfortable while their neck is rotated axially to the right as far as is comfortable.

The neck sagittal motion (left) 1380 trial is used to assess dynamic mechanical characteristics of neck motion in the sagittal plane when rotated axially to the left. For this cervical spine motion, the individual patient, employee, or subject 115 is instructed to tilt their head forward and back repeatedly as fast as is comfortable while their neck is rotated axially to the left as far as is comfortable.

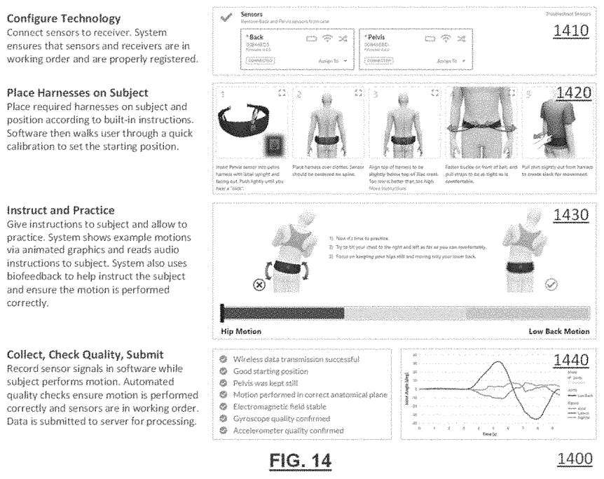

FIG. 14 is an illustration of an example functional assessment 311 data collection workflow 1400. In this workflow, a technology platform 120 user interface 451 is used to guide the user 110 and individual patient, employee, or subject 115 through one or more functional assessment 311 protocols similar, but not limited to, to those outlined above (e.g., low back motion assessment protocol 1200 or neck motion assessment protocol 1300). General steps include configuring any sensors or other technology required to capture data 1410, placing harnesses 1420 on the individual patient, employee, or subject 115, providing instructions to the patient employee, or subject 115 and allowing time to practice 1430, collecting data, checking data quality 1440, and submitting results for processing. Animations, graphics, computer-read instructions, biofeedback applications, and other modalities may be used to help communicate the protocol to the individual patient, employee, or subject 115 and ensure data quality.

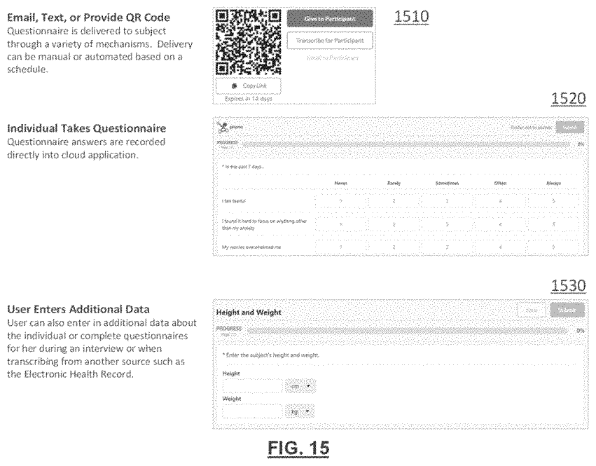

FIG. 15 is an illustration of an example biopsychosocial questionnaire data collection workflow 1500. In this workflow, a technology platform 120 user interface 451 is used to email, text, or provide a QR code 1510 for one or more digital questionnaires to an individual patient, employee, or subject 115. Questionnaires may also be sent automatically on a predefined schedule (e.g., every month or 3 months after a specific event). The individual patient, employee, or subject 115 then takes the one or more questionnaires 1520 and data is stored directly into the cloud web application 450. The user 110 may also enter additional information about the individual patient, employee, or subject 115 or complete questionnaires for her during an interview or when transcribing from another source such as an electronic health record 1530.

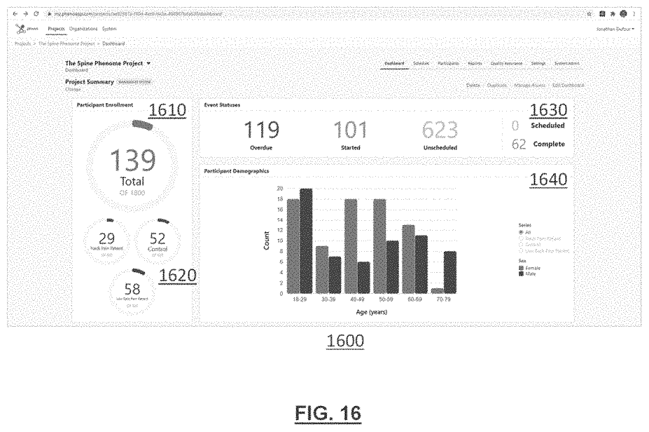

FIG. 16 is an illustration of an example project summary report dashboard 1600 that helps users track subject enrollment and demographics. In this example, total enrollment into a specific project is shown at 1610, as well as enrollment broken down by cohort (e.g., control, low back pain patient, neck pain patient) at 1620. The status (e.g., overdue, started, unscheduled, scheduled, complete) of all events is displayed at 1630, and distributions of enrolled individual patient, employee, or subject 115 demographics (e.g., age, sex) are shown 1640.

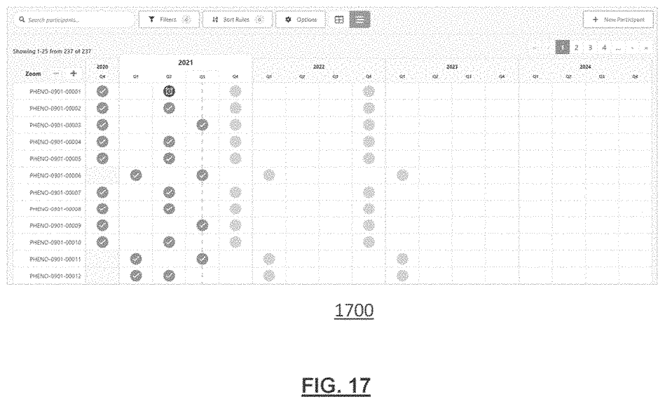

FIG. 17 is an illustration of an example subject summary report dashboard 1700 that helps users 110 track event completion progress and overall subject journey. Users 110 can quickly view which past, present, or future events need immediate attention to help optimize operations and guide communications between the user 115 and individual patients, employees, or subjects 115.

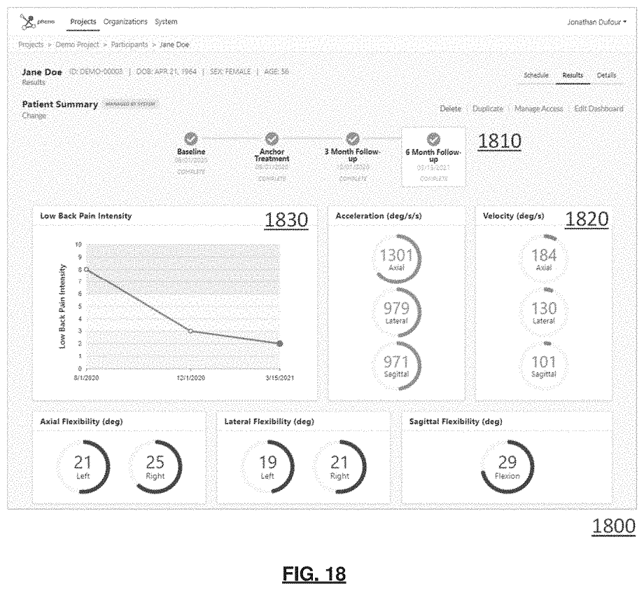

FIG. 18 is an illustration of an example individual patient, employee, or subject 115 summary report dashboard 1800 showing processed feature and characteristic measurements for motion capabilities and pain. The dashboard displays all events and their completion statuses at 1810 and can display both cross-sectional data at 1820 and historic data at 1830 to help understand whether a specific biomarker is improving or worsening. Contextual data such as thresholds, targets, goals, may also be displayed. Other data and data visualization formats may be included. All report dashboards are flexible and may be comprised of one or more windows, infographics, or charts that the user 110 may customize to view data.

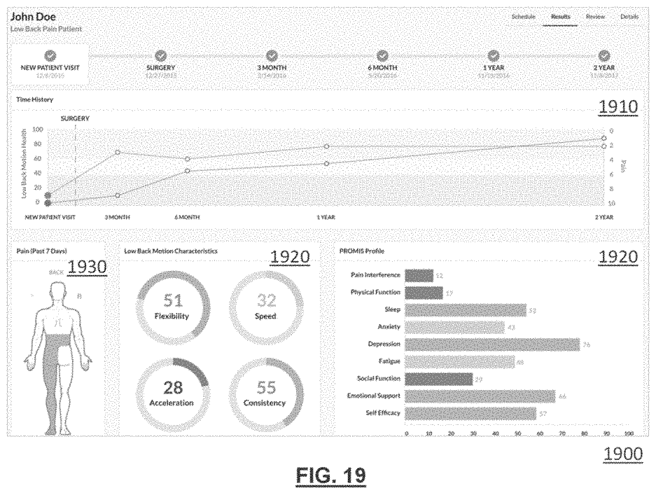

FIG. 19 is an illustration of an example individual patient, employee, or subject 115 summary report dashboard 1900 showing composite biomarkers at 1910, as well as normalized (e.g., t-scores) feature and characteristics for motion capabilities, pain, and other biopsychosocial biomarkers at 1920. Pain body maps are also presented at 1930. Other data and data visualizations may be included.

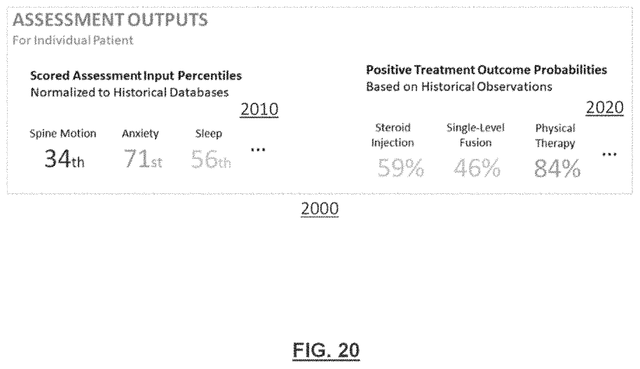

FIG. 20 is an illustration of an example individual patient, employee, or subject 115 summary report dashboard 2000 showing composite biomarker percentiles 2010 and modeled treatment outcome probabilities 2020. Relationships to composite biomarker distributions may also be represented by t-scores or other statistical methods and may be referenced based on one or more reference populations. Composite biomarkers may be derived for a specific domain (e.g., depression, fatigue, social function) or may be derived from multiple domains. Treatment outcome probabilities may be referenced based on positive, null (no change), or negative outcomes. In this example, positive treatment outcome probabilities for three medical procedures (e.g., steroid injection, single-level fusion, and physical therapy) are presented.

For spine disorder patients, outcome probabilities for a variety of medical treatments and procedures may be produced such as for medications, spinal manipulations or chiropractic care, passive (e.g., ultrasound, TENS, diathermy) and/or active (e.g., supervised exercise, aquatic therapy) physical therapy, massage therapy, home-based exercise programs (unsupervised or supervised), acupuncture, cognitive behavioral therapy, mindfulness, meditation, yoga, diet or nutrition programs, weight loss programs, injections, radiofrequency ablations, peripheral nerve stimulators, spinal cord or dorsal root stimulators, and spine surgery (e.g., decompression with or without instrumentation, arthroplasties).

For employees at risk for injury, outcome probabilities may be produced based on occupational interventions such as training programs, engineering controls, changes to work environments, or the introduction of new equipment

Other treatments, interventions, jobs, activities, or events may be the subject of outcome probability predictions.

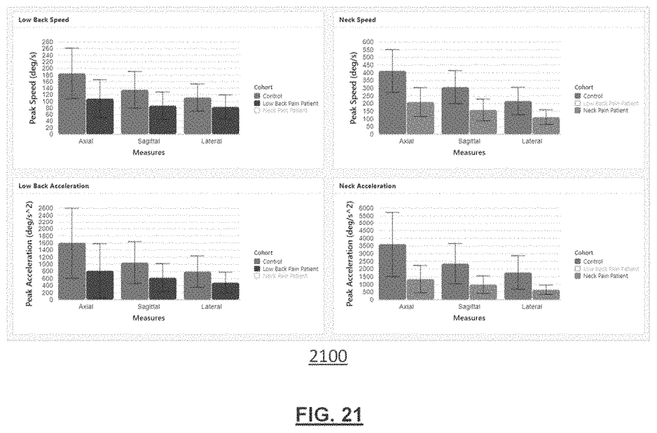

FIG. 21 is an illustration of an example population summary report dashboard 2100 showing differences between cohorts for specific motion assessment feature biomarkers (e.g., speed, acceleration). Other data or data visualization methods may be included.

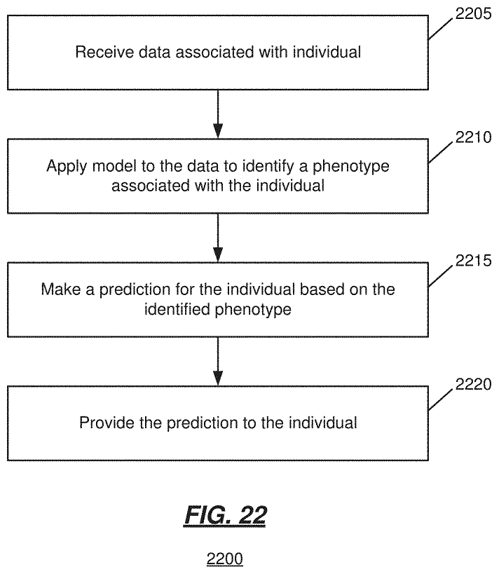

FIG. 22 is an operational flow of an implementation of a method 2200 for generating one or more predictions based on user data. The method 2200 may be implemented by the technology platform 120.

At 2205, an individual patient, employee, or subject's 115 data is received. The data 130 may be received by the technology platform 120. The data 130 may include data collected from one or more sensors (e.g., wearable sensors), data collected from one or more questionnaires (e.g., biopsychosocial data), and medical history data of the user (e.g., medical records). Depending on the embodiment, the user 110 of the technology platform 120 may be a healthcare provider trying to determine the best treatment plan for a medical condition, or an employer trying to learn how to avoid workplace injuries or to improve workplace health.

At 2210, a model is applied to the collected data 130 to identify a phenotype associated with the individual patient, employee, or subject 115. The model may be applied to some or all of the collected data 130 by the technology platform 120. Depending on the embodiment, the model may have been trained using records from a historical reference database using artificial intelligence or machine learning based techniques.

At 2215, a prediction for the individual patient, employee, or subject 115 is made based on the identified phenotype. The prediction may be made by the technology platform 120. Where the user 110 is a healthcare provider, the prediction may be for the effectiveness of one or more treatment plans or medical procedures contemplated by the provider and patient. Where the user 110 is an employer, the prediction may be a prediction related to one or more injuries that may be sustained by the employee or group of employees.

At 2220, the prediction is provided to the user 110 and/or the individual patient, employee, or subject 115. The prediction may be provided to the user 110 in one or more reports or dashboards associated with the user. Where the user 110 is a healthcare provider, the prediction may further be provided to one or more other medical professionals (e.g., doctors, nurses, and physical therapists) that are treating the patient. Where the user 110 is an employer, the prediction may be provided to one or more supervisors who may use the prediction to assess employee working conditions and/or employee safety.

The technology platform 120 described herein may provide a variety of use cases. One such use cases is as a low-risk and low-cost decision-support system that grants healthcare providers access to a holistic array of patient-specific biopsychosocial biomarkers that can be used to aid in the practice of personalized medicine. The biomarkers extracted from patient data provide insights into how a patient is faring in a particular biopsychosocial domain (neuromusculoskeletal biomechanics, physical function, sleep, fatigue, anxiety, depression, etc.) relative to large normative reference databases. These biomarkers for a patient can be tracked quantitatively over time to measure the patient's response to treatment interventions. Additionally, the platform 120 is positioned to use historical outcome observations from large reference databases in combination with machine learning and artificial Intelligence to predict which treatment options are most likely to be effective for a particular patient.

Another use case for the platform 120 is as a high-fidelity diagnostic platform for mechanical spine issues through sophisticated biomechanical spine models that are patient-specific and mechanistic in nature. These models ingest a variety of biomedical imaging, muscle activation, external force, and other data to calculate personalized spinal tissue forces. Once built, these models may be used to understand the mechanical impact of degenerative or surgical changes to the spine system. This information can be used to predict patient-specific surgical outcomes, evaluate the efficacy of new medical devices, or create a highly accurate physical spine model via a 3D printer for surgical planning or patient education.

Another use case for the platform 120 is as a population health management tool for employers, managed care organizations, or insurance providers. For example, employees for an employer may wear sensors, such as spine and back sensors, while they perform their employment duties. At the same time, employees may provide biopsychosocial data through one or more questionnaires. The collected data for the employees may be used by the platform 120 to identify activities performed by the employees that may be leading to medical conditions, as well as certain negative biomarkers that may be prevalent among employees (e.g., depression or anxiety). These identified activities and/or biomarkers may be identified in a report that is presented to the entity. The report may include a quantification of the levels of risk associated with various physical exposure and other biopsychosocial factors by comparing observed measurements to known thresholds. With this information, the user may identify which occupational factors are contributing most to injuries and how much those factors need to be changed to realize a reduction injuries. This information may help inform resource allocation decisions with regards to improving overall safety and preventing injuries in the workplace. After interventions have been implemented, the technology platform 120 can also periodically retest the employees to determine if any of the changes have led to improvements in injury rates or overall risk.

Another use case for the platform 120 is as a fully integrated research platform that enables researchers to design studies, capture a wide range of relevant biomarkers, track study progress, analyze data (e.g., historical patient data), and generate visual dashboards and reports in one central system. The platform 120 may be designed to meet 21 CFR Part 11 compliance requirements, making it useful for conducting studies on investigational devices.

Another use case for the platform 120 is as a tool for facilitating provider-patient engagement by setting and working towards quantitative goals, educating patients on their condition, and validating their experience with chronic low back and neck pain. For example, a patient may use a dashboard to view their progress with respect to certain biomarkers including pain, and to communicate with their provider.

Numerous other general purpose or special purpose computing devices environments or configurations may be used. Example computing devices, environments, and/or configurations that may be suitable for use include, but are not limited to, personal computers, server computers, cloud-based systems, handheld or laptop devices, multiprocessor systems, microprocessor-based systems, network personal computers (PCs), minicomputers, mainframe computers, embedded systems, distributed computing environments that include any of the above systems or devices, and the like.

Computer-executable instructions, such as program modules, being executed by a computer may be used. Generally, program modules include routines, programs, objects, components, data structures, etc. that perform particular tasks or implement particular abstract data types. Distributed computing environments may be used where tasks are performed by remote processing devices that are linked through a communications network or other data transmission medium. In a distributed computing environment, program modules and other data may be located in both local and remote computer storage media including memory storage devices.

Although exemplary implementations may refer to utilizing aspects of the presently disclosed subject matter in the context of one or more stand-alone computer systems, the subject matter is not so limited, but rather may be implemented in connection with any computing environment, such as a network or distributed computing environment. Still further, aspects of the presently disclosed subject matter may be implemented in or across a plurality of processing chips or devices, and storage may similarly be implemented across a plurality of devices. Such devices might include personal computers, network servers, and handheld devices, for example.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

What is claimed:

1. A method comprising:
receiving data associated with an individual by a computing device;
applying a model to the individual's data to identify a phenotype associated with the individual by the computing device;
making a prediction for the individual based on the identified phenotype by the computing device; and
providing the prediction to the individual or an agent of the individual by the computing device.

2. The method of claim 1, wherein receiving data comprises receiving the individual's data from one or more sensors worn by the individual.

3. The method of claim 2, wherein the sensor comprises one or more inertial measurement unit (IMU) sensors.

4. The method of claim 1, wherein receiving data comprises receiving medical history data for the individual.

5. The method of claim 1, wherein receiving data comprises receiving biopsychosocial biomarkers for the individual.

6. The method of claim 1, wherein receiving data comprises receiving data from one or more digital questionnaires completed by the individual.

7. The method of claim 1, wherein the identified phenotype is derived from one or more biomarkers that is an indicator of dynamic low back motion function.

8. The method of claim 1, wherein the identified phenotype is derived from one or more biomarkers that is an indicator of dynamic neck motion function.

9. The method of claim 1, wherein the prediction comprises a predicted success likelihood for a medical procedure.

10. The method of claim 1, wherein the prediction comprises an injury likelihood or injury for the individual.

11. The method of claim 1, wherein the prediction comprises an injury likelihood for a group of individuals.

12. The method of claim 1, wherein the individual is a patient or an employee.

13. The method of claim 1, further comprising:
receiving a historical reference database comprising a plurality of records;
for each record, identifying unique biomarkers associated with the record; and
training the model using the plurality of records and biomarkers to identify unique phenotypes.

14. A technology platform for providing patient care, injury prevention, or research services comprising:
at least one computing device; and
a computer-readable medium with computer-executable instructions stored thereon that when executed by the at least one computing device cause the at least one computing device to:
receive data associated with an individual;
apply a model to the individual's data to identify a phenotype associated with the individual;
make a prediction for the individual based on the identified phenotype; and
provide the prediction to the individual or an agent of the individual.

15. The technology platform of claim 14, further comprising computer-executable instructions stored thereon that when executed by the at least one computing device cause the at least one computing device to:
receive the user data from one or more sensors worn by the individual.

16. The technology platform of claim 15, wherein the sensor comprises an inertial measurement unit (IMU) sensor.

17. The technology platform of claim 14, wherein the received data comprises medical history data for the individual.

18. The technology platform of claim 14, wherein the received data comprises data from one or more digital questionnaires completed by the individual.

19. The technology platform of claim 14, wherein the prediction comprises a predicted success likelihood for a medical procedure.

20. The technology platform of claim 14, wherein the prediction comprises an injury likelihood or injury risk for the user.
